(19) United States
(12) Patent Application Publication
(10) Pub. No.: US 2008/0025583 A1
(43) Pub. Date: Jan. 31, 2008

(54) SYSTEM AND METHOD FOR ON-DEMAND VISUAL ENHANCEMENT OF CLINICAL CONITIONS IN IMAGES

(75) Inventors: Kadri Nizar Jabri, Waukesha, WI (US); Renuka Uppaluri, Pewaukee, WI (US); Gopal B. Avinash, New Berlin, WI (US); Saad Ahmed Sirohey, Pewaukee, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/460,315

(22) Filed: Jul. 27, 2006

(51) Int. Cl. A61B 5/05

(52) U.S. Cl. 382/128; 600/407

(57) ABSTRACT

A system and method for enhancing visualization of clinical conditions comprising receiving imaging data on a subject from an imaging modality, receiving user input on at least one suspected clinical condition of the subject undergoing imaging on the imaging modality, and processing the imaging data in association with a knowledgebase using an optimal image processing algorithm to enhance visualization of the at least one suspected clinical condition in at least one image.

### BACKGROUND OF THE INVENTION

The present invention relates generally to imaging systems, such as medical diagnostic imaging systems, and more particularly to a system and method for on-demand visual enhancement of clinical conditions in medical images.

Medical diagnostic imaging systems encompass a variety of imaging modalities, such as X-ray systems, computerized tomography (CT) systems, ultrasound systems, magnetic resonance (MR) systems, positron emission tomography (PET) systems, nuclear medicine systems, and the like. Medical diagnostic imaging systems generate images of an object, such as a patient, for example, through exposure to an energy source, such as X-rays passing through a patient. The generated images may be used for many purposes. For instance, internal defects in an object may be detected. Additionally, changes in internal structure or alignment may be determined. Fluid flow within an object may also be represented. Furthermore, the generated images may show the presence or absence of a particular clinical condition in a patient undergoing imaging. The information gained from imaging has applications in many fields, including medicine, manufacturing and security.

The current workflow of medical diagnostic imaging systems, specifically digital radiography systems including computed radiography systems, is for the acquired image to be processed by a single preferred set of image processing algorithms and image processing parameters at the acquisition or modality workstation. The processed image is then typically sent to a picture archival communication system (PACS) for review by a radiologist. Therefore, as a result of this workflow, the flexibility of post-processing of an image after receipt by PACS is very limited.

Image processing algorithms are usually intended to enhance overall image attributes (edge sharpness, contrast, etc.) rather than clinical-condition specific attributes (lung nodules, rib fractures, etc.). Image processing parameters are therefore usually tuned to give the radiologist his or her preferred overall image “look” for each imaged anatomy. As a result, the processing parameters of a preferred image “look” may not be optimal for enhancing any clinical condition present in an image. Therefore, it is desirable to develop images with multiple clinical-condition specific “looks” for the purpose of enhancing the visualization of clinical conditions in the images.

The current methodology for developing image processing algorithms in digital radiography systems is to develop and tune algorithms for specific conditions, both clinical and imaging. Currently, developers generally write unique software programs to generate results for numerous specific clinical conditions. This requires a unique software program be generated for each specific clinical condition. To enhance a specific clinical condition in an acquired image, the acquired image would be processed with only one clinical-condition specific algorithm. In this case, the usefulness of the enhanced visualization is only applicable when the images contain the target clinical condition. Since radiography is frequently used as a screening method for a very large number of clinical conditions, this approach is of limited clinical value. The above approach creates an added burden on the software developers as well as the clinicians. Utilizing unique algorithms for specific conditions is generally inefficient and prohibitively expensive for development and commercialization.

Another possible method for enhancing the visualization of clinical conditions in images is to process the acquired images with multiple clinical-condition specific algorithms, thereby creating multiple processed images for review on PACS. This would require the development of unique algorithms for every single clinical condition scenario. This is counter productive as it becomes prohibitively expensive for development, validation, commercialization, and regulatory clearance, etc. This approach places a significant strain on workflow and efficiency, making it unwieldy in the current radiology practice environment where radiologists often are under very stringent time constraints. Even if the data overload and efficiency requirements are overlooked, it is still a challenging problem to develop techniques for enhancing the visualization of multiple clinical conditions in images.

Therefore, a need exists for a system and method for providing on-demand enhancement of clinical conditions in images that may be utilized to optimally select a computer algorithm, or path of algorithms, based on input. Such a system and method may utilize anatomical, clinical and image acquisition conditions and scrutinize selection of algorithms and parameters for a given clinical purpose.

### BRIEF DESCRIPTION OF THE INVENTION

In an aspect, a method for enhancing visualization of clinical conditions comprising receiving imaging data on a subject from an imaging modality, receiving user input on at least one suspected clinical condition of the subject undergoing imaging on the imaging modality, and processing the imaging data in association with a knowledgebase using an optimal image processing algorithm to enhance visualization of the at least one suspected clinical condition in at least one image.

In another aspect, a method for enhancing visualization of a clinical condition in a medical image comprising receiving clinical data on a subject undergoing imaging on an imaging modality, acquiring imaging data on the subject from the imaging modality, and processing the clinical data and the imaging data in association with a knowledgebase using an optimal image processing algorithm with optimal parameter settings for enhancing visualization of at least one clinical condition in at least one image.

In yet another aspect, a system for enhancing visualization of clinical conditions comprising an input for receiving imaging data on a subject from an imaging modality, a user interface for receiving user input on at least one suspected clinical condition of the subject undergoing imaging on an imaging modality, and a processor coupled to the input and the user interface for processing the imaging data in association with a knowledgebase using an optimal image processing algorithm to enhance visualization of the at least one suspected clinical condition in at least one image.

In still yet another aspect, a system for enhancing visualization of clinical conditions comprising an acquisition workstation coupled to and receiving imaging data on a patient from an imaging modality, the acquisition workstation including a user interface for performing on-demand selection of at least one clinical condition to be enhanced in at least one image, and a computer coupled to the input and the user interface with at least one computer-usable medium having computer readable instructions stored thereon for execution by a processor, the computer performing a method comprising accessing clinical data on the patient undergoing imaging, receiving imaging data from the imaging modality, and processing the clinical data and the imaging data in association with a knowledgebase using an optimal image processing algorithm with optimal parameter settings to enhance visualization of a selected clinical condition in an image.

In a further aspect, a computer program product for use with a computer, the computer program product comprising a computer-usable medium having computer readable instructions stored thereon for execution by a processor, the computer readable instructions comprising an accessing routine for accessing clinical data on a subject undergoing imaging on an imaging modality, a receiving routing for receiving imaging data on the subject from the imaging modality, and a processing routine for processing the clinical data and the imaging data in association with a knowledgebase using an optimal image processing algorithm with optimal parameter settings to enhance visualization of at least one clinical condition in at least one image.

Various other features, objects, and advantages of the invention will be made apparent to those skilled in the art from the accompanying drawings and detailed description thereof.

### BRIEF DESCRIPTION OF THE DRAWINGS

FIG. 1 is a block diagram of a system used in accordance with an embodiment of the present invention;

FIG. 2 is a block diagram of a system used in accordance with another embodiment of the present invention;

FIG. 3 is a flow diagram of a process used in accordance with an embodiment of the present invention;

FIG. 4 is a flow diagram of a process used in accordance with another embodiment of the present invention;

FIG. 5 is a flow diagram of a process used in accordance with yet another embodiment of the present invention;

FIG. 6 is a diagram of an algorithm selection process used in accordance with an embodiment of the present invention; and

FIG. 7 is a table illustrating an example of a knowledgebase used in accordance with an embodiment of the present invention.

### DETAILED DESCRIPTION OF THE INVENTION

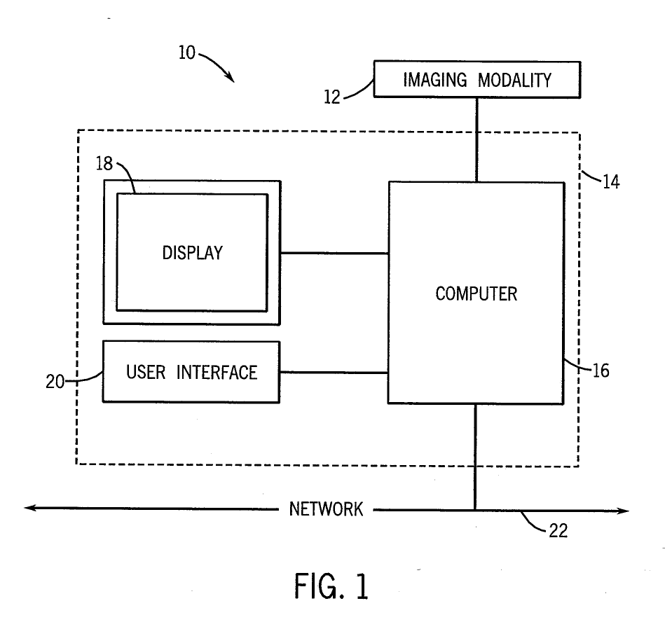

Referring now to the drawings, FIG. 1 illustrates a block diagram of an embodiment of a system 10 for acquiring, manipulating, processing and displaying medical images. The system is designed for enhancing the visualization of clinical conditions in medical images. The system 10 includes an acquisition workstation 14 coupled to and receiving imaging data on a subject from an imaging modality 12. The acquisition workstation 14 includes at least one computer 16 coupled to at least one display 18 and at least one user interface 20. The at least one computer 16 may be any piece of equipment with software that permits electronic medical images, such as X-rays, ultrasound, CT, MR, PET, or nuclear medicine images, for example, to be electronically acquired, processed, stored or transmitted for viewing and diagnostic operations. The at least one computer 16 includes at least one computer-usable medium having computer readable instructions stored thereon for execution by a processor. The computer readable instructions include a plurality of algorithms for enhancing at least one clinical condition on at least one image of a subject undergoing imaging on the imaging modality and a rules engine for determining the optimal image processing algorithm and associated parameters for enhancing visualization of the suspected or selected clinical condition. The at least one display 18 may include multiple displays or multiple display regions on a screen. Accordingly, any number of displays may be utilized in accordance with the present invention. The display 18 may display a list of clinical conditions to select from using the at least one user interface 20. The at least one user interface 20 receives inputs from a user for performing on-demand selection of at least one clinical condition to be enhanced in at least one image. The inputs may be the selection of a suspected clinical condition of a subject undergoing imaging on the imaging modality. The user interface 20 provides for on-demand image processing selection by a user. The acquisition workstation 14 may be coupled to a network 22 physically, by wire, or through a wireless medium.

In another embodiment, the acquisition workstation 14 comprises at least two inputs and at least one output. One input is for receiving imaging data on a subject from the imaging modality 12 and a second input is for receiving clinical data on the subject and a knowledgebase from the network 22. The at least one output is for sending data to the network 22. The acquisition workstation 14 comprises at least one computer 16 coupled to at least one imaging modality, at least one display 18 and at least one user interface 20. The computer 16 includes at least one storage device for storing the clinical data, the imaging data and the knowledgebase. The at least one computer 16 processes the imaging data in association with a knowledgebase using an optimal image processing algorithm to enhance visualization of the at least one suspected clinical condition in at least one image. The at least one display 18 displays the enhanced visualization of the at least one suspected clinical condition in the at least one image. The user interface 20 receives user input on at least one suspected clinical condition of the subject undergoing imaging on the imaging modality.

In yet another embodiment, a computer program product for use with a computer, the computer program product comprising a computer-usable medium having computer readable instructions stored thereon for execution by a processor, the computer readable instructions comprising an accessing routine for accessing clinical data on a subject undergoing imaging on an imaging modality, a receiving routing for receiving imaging data on the subject from the imaging modality, and a processing routine for processing the clinical data and the imaging data in association with a knowledgebase using an optimal image processing algorithm with optimal parameter settings to enhance visualization of at least one clinical condition in at least one image.

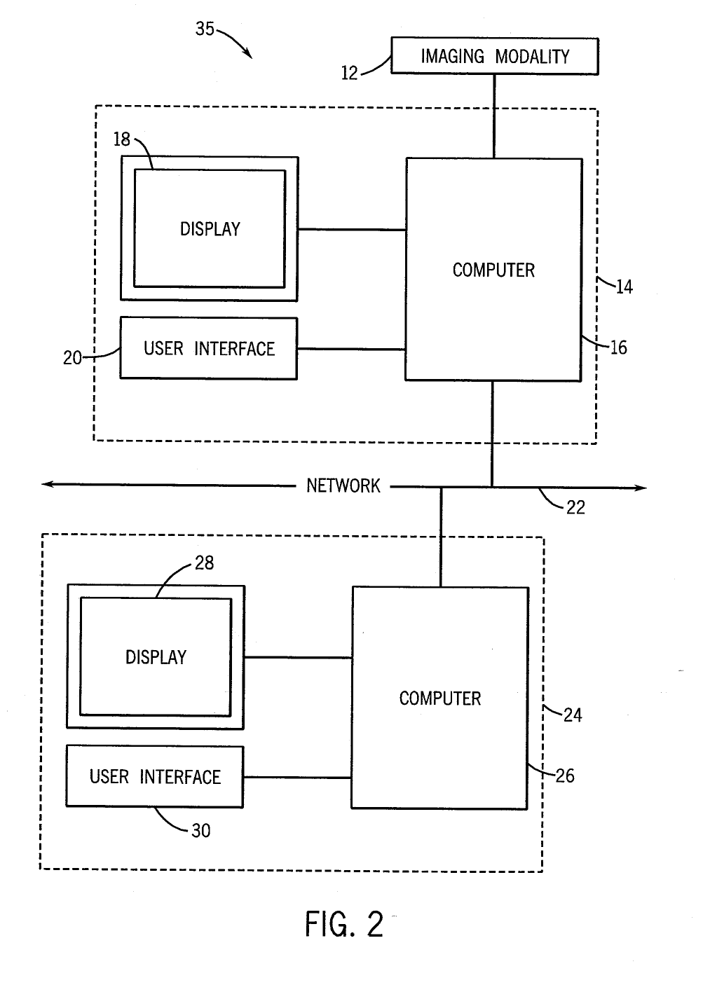

The acquisition workstation 14 coupled to the imaging modality 12 and the network 22 may be coupled to at least one diagnostic workstation 24 as is shown in the embodiment of FIG. 2. FIG. 2 illustrates a block diagram of another embodiment of a system for acquiring, manipulating, processing and displaying medical images. Coupled to the network 22 is a diagnostic workstation 24. The diagnostic workstation 24 may be part of a picture archival communication system (PACS). A PACS typically includes equipment and software that permits images to be electronically acquired, stored, transmitted and viewed. Users, such as radiologists, may view images on diagnostic workstations and execute computer assisted detection and diagnostic tasks.

As shown in FIG. 2, the system comprises at least one diagnostic workstation, such as a PACS, coupled to the network 22 for reviewing the enhanced visualization of the at least one suspected clinical condition in at least one image. The diagnostic workstation 24 includes at least one computer 26 coupled to at least one display 28 and at least one user interface 30.

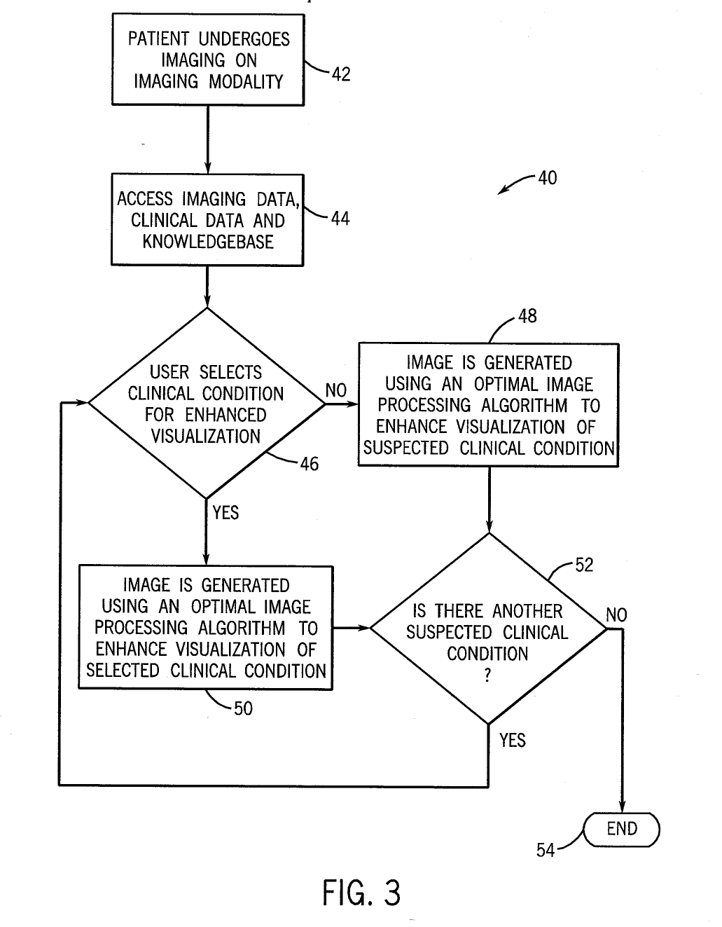

FIG. 3 illustrates an embodiment of a method 40 for selecting a computer algorithm for processing a medical image. The method 40 is designed for enhancing the visualization of clinical conditions in medical images. The images can be of any dimension (2D, 3D, 4D, etc). A patient undergoes imaging on an imaging modality 42. Imaging data on the patient is received or accessed from the imaging modality 44. In addition, clinical data on the patient and data from a knowledgebase is also received or accessed 44. A user may select at least one suspected clinical condition of the patient undergoing imaging on the imaging modality for enhanced visualization 46. A plurality of specific clinical conditions for visual enhancement (set of clinical-condition specific “looks”) are offered at the acquisition workstation, typically from a map, list, free form, etc., based on present patient conditions, patient history, physical information and imaging data that is available as inputs to the system. This set of clinical-condition specific “looks” can be comprehensive or automatically generated based on patient history and/or suspect clinical condition. The user is given the option to create one or more processed images based on suspect clinical condition by selecting the clinical-condition specific “looks” through the user interface. If the user does not select a clinical condition for enhancement, in image is automatically generated with enhanced visualization of a suspected clinical condition from the clinical data, prior medical history of the subject, and/or knowledgebase data 48. If the user does select a clinical condition for enhancement, an image is generated with enhanced visualization of the user selected clinical condition 50. The imaging data and clinical data are processed in association with the knowledgebase using an optimal image processing algorithm to enhance visualization of the at least one suspected and/or selected clinical condition in at least one image. The process of a user selecting a clinical condition for enhanced visualization 46 and the process of generating the image 48, 50 can be repeated any number of times for a plurality of suspected clinical conditions. After an image is generated 48, 50, if there is another suspected clinical condition 52, then the process jumps back to the user selecting another clinical condition for enhanced visualization 46 and new images are generated 48, 50. If there is not another suspected clinical condition, then the process ends 54. The optimal image processing algorithm includes one or more of detection, segmentation, registration, and enhancement of the at least one clinical condition. The imaging data includes imaging type, protocol and/or technique information. The imaging data also includes images whose acquisition technique was optimized for detecting a specific clinical condition. The clinical data includes a repository of the subject's medical data, including the subject's personal medical history, current physical state and/or present medical condition. The clinical data may also include an electronic medical record (EMR) of the subject. The knowledgebase includes a plurality of clinical conditions, and a plurality of associated algorithms and a plurality of algorithm parameters for the plurality of clinical conditions.

In another embodiment, a patient undergoes imaging on an imaging modality and an image is generated using an image processing algorithm. The acquired images are processed using a default “standard” look, whereby no specific clinical condition is necessarily enhanced. This is normal workflow and requires no explicit action by the user. The system then accesses the imaging data, clinical data and knowledgebase. A user may select a clinical condition for enhanced visualization. A new image is generated using an optimal image processing algorithm to enhance visualization of the suspected and/or selected clinical condition. All processed images, standard plus clinical condition enhanced, are sent to a diagnostic workstation for final review by radiologists.

For the above embodiments, if imaging data acquired during a patient exam is tagged for follow-up, the clinical-condition specific visual enhancement algorithm chosen may be based on the previous exam so that no additional user input may be required. However, if additional clinical conditions need to be visually enhanced, the user can intervene and provide additional input. The follow-up exam will be part of the clinical input for the imaging system and method.

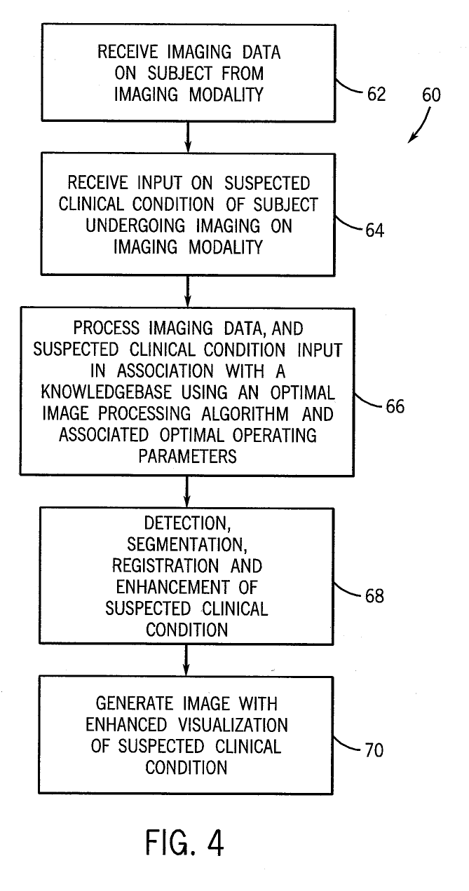

FIG. 4 is a flow diagram of another embodiment of a method 60 for enhancing the visualization of clinical conditions in medical images. The images can be of any dimension (2D, 3D, 4D, etc). The method 60 includes selecting an optimal computer algorithm and associated parameters for enhancing the visualization of clinical conditions in images. The method 60 may select an optimal computer algorithm based on values of several inputs. These inputs include imaging data, clinical data, and structured knowledgebase information. The imaging data may include the image of the anatomy and associated parameters as well as image meta-data. The image meta-data may include image acquisition information, such as, for example, modality and slice thickness. The clinical data may include clinical purpose information, for example, task information such as an examination to determine whether a patient has cancer in the lung. Based on the imaging data and clinical data, an optimal computer algorithm may be selected to achieve visual enhancement of a suspect clinical condition. The optimal computer algorithm may be selected from a structured knowledgebase having structured knowledgebase information. A structured knowledgebase may be a database or server having information to select the optimal computer algorithm to achieve a given clinical purpose based on the input. Once the optimal computer algorithm is selected, the imaging data may be processed by the optimal computer algorithm with associated parameters.

The method 60 includes receiving imaging data on a subject from an imaging modality 62. The method 60 also includes receiving at least one input on a suspected clinical condition of the subject undergoing imaging on the imaging modality 64. The method further includes processing the imaging data and suspected clinical condition input in association with a knowledgebase using an optimal image processing algorithm with optimal parameter settings for enhancing visualization of at least one clinical condition in at least one image 66. The optimal image processing algorithm includes one or more of detection, segmentation, registration, and enhancement of the at least one clinical condition 68. An image is generated with enhanced visualization of a suspected clinical condition of the subject 70.

The method 60 further comprises a user selecting at least one clinical condition for enhanced visualization in at least one image at a user interface. The at least one clinical condition for enhanced visualization is selected by a user from a list, map, free form, etc., of clinical conditions presented to the user at the user interface. The at least one clinical condition for enhanced visualization is selected automatically by a selection algorithm based on the subject's prior medical history and/or suspect clinical condition.

In the embodiments described above, the optimal image processing algorithm includes one or more of detection, segmentation, registration, and enhancement of the at least one clinical condition. The imaging data includes imaging type, protocol and/or technique information. The imaging data also includes images whose acquisition technique was optimized for detecting a specific clinical condition. The knowledgebase includes a plurality of clinical conditions, and a plurality of associated algorithms and a plurality of algorithm parameters for the plurality of clinical conditions.

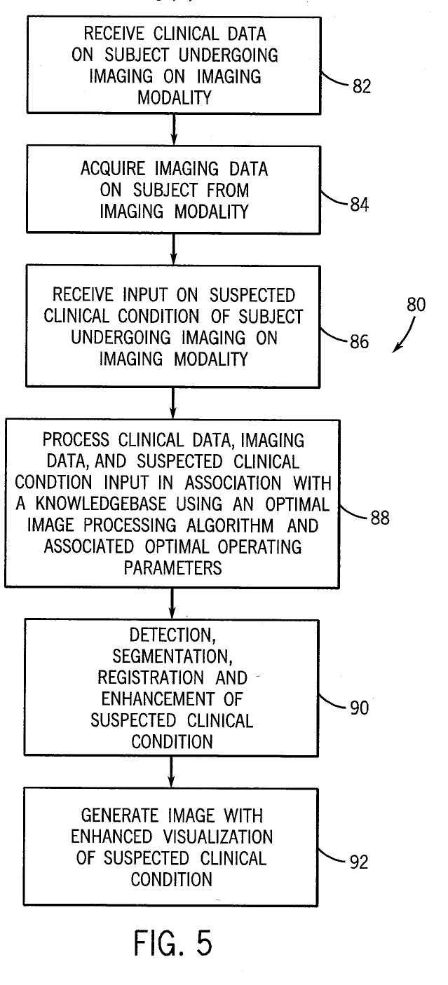

FIG. 5 is a flow diagram of yet another embodiment of a method 80 for enhancing the visualization of clinical conditions in medical images. The images can be of any dimension (2D, 3D, 4D, etc). The method 80 includes receiving clinical data on a subject undergoing imaging on an imaging modality 82. The clinical data includes a repository of the subject's medical data, including the subject's personal medical history, current physical state and/or present medical condition. The clinical data may also include an electronic medical record (EMR) of the subject. The method 80 also includes acquiring imaging data on the subject from the imaging modality 84. The method 80 further includes receiving at least one input on a suspected clinical condition of the subject undergoing imaging on the imaging modality 86. The method 80 further includes processing the clinical data, imaging data and suspected clinical condition input in association with a knowledgebase using an optimal image processing algorithm with optimal parameter settings for enhancing visualization of at least one clinical condition in at least one image 88. The optimal image processing algorithm includes one or more of detection, segmentation, registration, and enhancement of the at least one clinical condition 90. An image is generated with enhanced visualization of a suspected clinical condition of the subject 92.

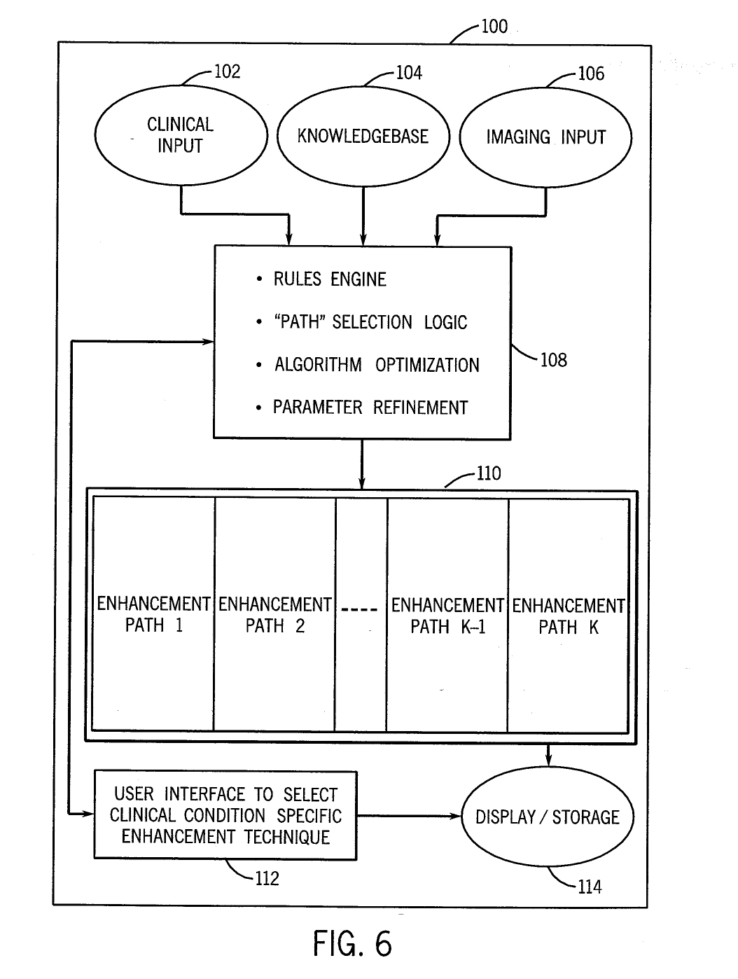

FIG. 6 is a diagram of an embodiment of an algorithm selection process 100 for visually enhancing clinical conditions in medical images. The process 100 includes receiving or acquiring data from three inputs. The three inputs are clinical data on a subject from a clinical input 102, imaging data on the subject from an imaging input 106, and information from a structured knowledgebase 104. These inputs are directed to a rules engine 108. The rules engine 108 represents at least one computer software program executed by a processor. The processor receives clinical data, imaging data, information from the structured knowledgebase, and clinical-condition specific selection data from a user interface in order to select optimal enhancement algorithm with optimal parameters. The rules engine 108 accesses clinical data, imaging data and information from a structured knowledgebase. The clinical data may include clinical purpose information, for example, body parts, disease type, tracers used, screening, follow-up, diagnostic rule out, or differential diagnostic information. The imaging data may include the image of the anatomy and associated parameters as well as image meta-data. The image meta-data may include image acquisition information, such as, for example, modality information, slice thickness, dose, reconstruction information, pulse sequences, weighting, etc. Both the clinical data and imaging data may reside on the computer and may be accessed accordingly by the computer software executing the method. Alternatively the clinical and imaging data may reside on a different computer unit, or different computer units, systems, databases, servers, or other storage or processing device and be accessed accordingly. A structured knowledgebase may be a database or server comprising a finite set of algorithms that span the possible algorithms for the clinical purpose. For example, the structured knowledgebase may be information about which computer algorithms are optimal to achieve a clinical task given a set of data and parameters. The structured knowledgebase information may be stored as part of computer, or may be stored in an external location, such as database, and connected to computer via a network. The user interface is provided for on-demand processing selection. The user is given the option to create one or more processed images based on a suspect clinical condition by selecting from a plurality of specific clinical conditions to be visually enhanced in the images through the user interface.

The rules engine 108 includes algorithm path selection logic for selecting the optimal enhancement algorithm with optimal parameters for processing at least one medical image with clinical condition enhancement. The rules engine 108 selects an optimal computer algorithm from a plurality of computer algorithms, based on the clinical input 102, image input 106, knowledgebase 104, and user input 112 on a suspect clinical condition. The rules engine 108 also performs algorithm optimization and parameter refinement by assigning the optimal parameters to the selected algorithm based on the above-mentioned data. Once the optimal computer algorithm is selected, the algorithm may be executed and the results may be displayed and/or stored as shown in block 114.

Block 110 represents the different algorithmic paths that may be selected. Block 110 represents a plurality of computer algorithms that may be utilized to perform visual enhancement of the clinical conditions. As shown in the block 110, the paths may include Enhancement Path 1-Enhancement Path K. Which paths are chosen from block 110 may be based on the data 102, 104, 106 for the block of possible paths for enhancement 110. As illustrated in block 114, once the algorithm has been selected and executed, the results may be displayed and/or stored.

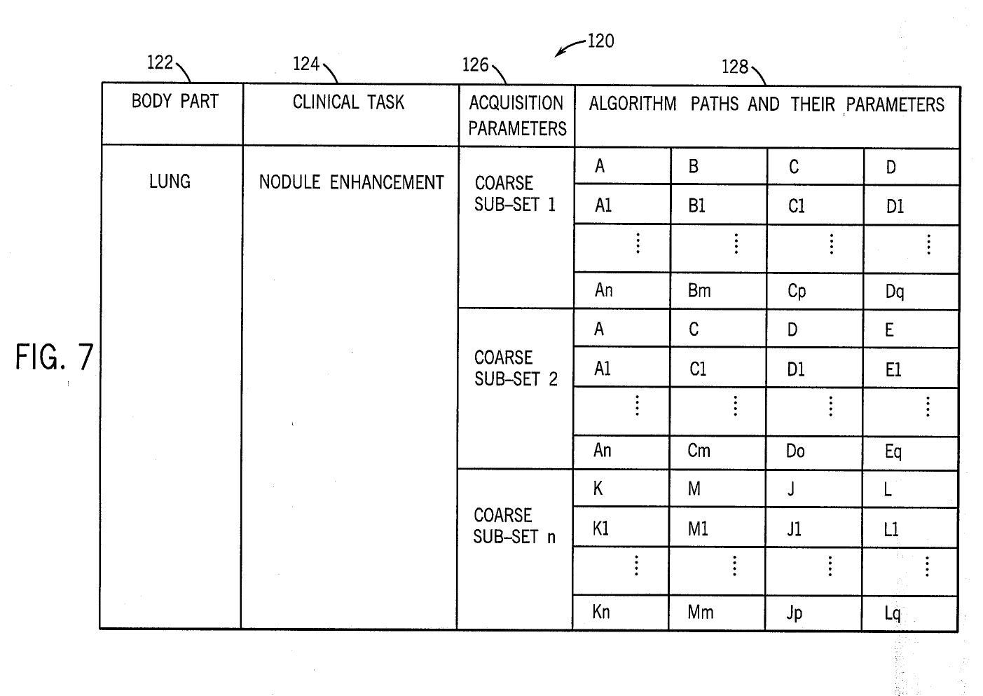

FIG. 7 illustrates an example table of fields that may be available in an example structured knowledgebase 120. Column 122 identifies a given body part. Column 124 identifies a given clinical task for the body part identified in column 122. Column 126 illustrates a plurality of piecewise linear sets. These sets include a range of acquisition parameters that have similar characteristics from a processing point of view.

Column 128 illustrates optimal computer algorithms for a given set of parameters. In an embodiment, depending on the parameters, a coarse sub-set may be selected, such as coarse sub-set 1, coarse sub-set 2, through coarse sub-set n. The coarse sub-sets identify different computer algorithms that may be executed to achieve the clinical purpose based on the imaging data and clinical data.

For the example shown in FIG. 7, the body part identified is the lung. If a user wishes to perform nodule sizing on the lung (i.e. the clinical purpose is to perform nodule sizing on the lung), various coarse sub-sets are identified. For example, coarse sub-set 1 through coarse sub-set n are shown in FIG. 7. Any number of coarse sub-sets may be used. A coarse sub-set may be selected based on the imaging data, for example the acquisition/reconstruction parameters. Each coarse sub-set has a computer algorithm that may be executed to achieve the clinical purpose. For example, if the acquisition/reconstruction parameters indicate that coarse sub-set 1 is optimal, algorithms A, B, C, or D may be selected. If coarse sub-set 2 is optimal, then algorithms A, C, D, or E may be selected. The selection of the algorithms may be determined by the imaging data and the clinical data. Continuing with the example, if the data and parameters indicate that the optimal algorithms to perform nodule sizing for a specific lung is path E in coarse sub-set 2, then coarse sub-set 2, algorithm E may be selected.

As an example, a patient is a scuba-diver complaining of severe chest pain after being involved in a diving accident. After acquiring a radiograph, the image is processed with the default “standard look.” Based on the patient's pain, as a clinical input, the case indicates the potential for a spontaneous pneumothorax, the technologist selects a “pneumothorax look” and creates an additional processed image that enhances the visualization of this clinical condition, if present. The radiologist receives the two processed images (“standard look” and “pneumothorax look”) on PACS for review. The pneumothorax is much more readily visualized in the version of the image processed with the “pneumothorax look” compared to the “standard look,” thereby improving diagnostic accuracy and potentially reducing the reading time. Being a pneumothorax patient, the person may be scanned every six hours. During the first scan, a user selects “pneumothorax look” based on suspicion. On subsequent scans, the system recognizes the patient name, ID, history and automatically processes the “pneumothorax look.”

In the example structured knowledgebase of FIG. 7, where the specific task of lung nodule enhancement is based on certain acquisition based criteria, associated multiple algorithmic paths and parameters are associated for each of the categories. An extension to the knowledgebase can be made for the variations caused by patient and/or clinical inputs. In the example described above of the pneumothorax patient, during the first scan, the user selects “pneumothorax look” based on suspicion. During subsequent exams when the clinical input is a follow-up exam, the user does not need to make a selection as the system recognizes the patient name, ID, history, and automatically processes the “pneumothorax look.”

In another embodiment, a computer program product for use with a computer, the computer program product comprising a computer-usable medium having computer readable instructions stored thereon for execution by a processor, the computer readable instructions comprising an accessing routine for accessing clinical data on a subject undergoing imaging on an imaging modality, a receiving routing for receiving imaging data on the subject from the imaging modality, and a processing routine for processing the clinical data and the imaging data in association with a knowledgebase using an optimal image processing algorithm with optimal parameter settings to enhance visualization of at least one clinical condition in at least one image.

The system and method utilizes clinical data and imaging data with prior knowledge to develop a rules engine that selects an optimal processing algorithm and parameters for disease specific feature enhancement in medical images.

A technical effect is that the system and method offers radiologists and other users enhanced visualization of a clinical condition when the patient history or physical condition indicates suspect clinical conditions, thereby potentially improving diagnostic accuracy. Another technical effect is that the system and method provides the ability to enhance images on-demand, in order to better detect certain clinical conditions without increasing reading time for images that do not have any suspected clinical condition.

In the embodiments described above, the system and method for on-demand visual enhancement of clinical conditions in images is designed to include enhancement of images in any dimensions, including but not limited to two-dimensional (2D) images, three-dimensional (3D) images, four-dimensional (4D) images, etc.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A method for enhancing visualization of clinical conditions comprising:
receiving imaging data on a subject from an imaging modality;
receiving user input on at least one suspected clinical condition of the subject undergoing imaging on the imaging modality; and
processing the imaging data in association with a knowledgebase using an optimal image processing algorithm to enhance visualization of the at least one suspected clinical condition in at least one image.

2. The method of claim 1, wherein the steps of receiving user input and processing the imaging data are repeated based on second and subsequent suspected clinical conditions.

3. The method of claim 1, wherein the imaging data includes imaging type, protocol and/or technique information.

4. The method of claim 1, wherein the imaging data includes images whose acquisition technique was optimized for detecting a specific clinical condition.

5. The method of claim 1, wherein the knowledgebase includes a plurality of clinical conditions, and a plurality of associated algorithms and a plurality of algorithm parameters for the plurality of clinical conditions.

6. A method for enhancing visualization of a clinical condition in a medical image comprising:
receiving clinical data on a subject undergoing imaging on an imaging modality;
acquiring imaging data on the subject from the imaging modality; and
processing the clinical data and the imaging data in association with a knowledgebase using an optimal image processing algorithm with optimal parameter settings for enhancing visualization of at least one clinical condition in at least one image.

7. The method of claim 6, further comprising selecting at least one clinical condition for enhanced visualization in at least one image.

8. The method of claim 7, wherein the at least one clinical condition for enhanced visualization is selected by a user from a list of clinical conditions presented to the user at a user interface.

9. The method of claim 7, wherein the at least one clinical condition for enhanced visualization is selected automatically by a selection algorithm based on the subject's clinical data, prior medical history and/or suspect clinical condition.

10. The method of claim 6, wherein the clinical data includes a repository of the subject's medical data, including the subject's personal medical history, current physical state and/or present medical condition.

11. The method of claim 6, wherein the clinical data includes an electronic medical record (EMR) of the subject.

12. The method of claim 6, wherein the optimal image processing algorithm includes one or more of detection, segmentation, registration, and enhancement of the at least one clinical condition.

13. The method of claim 6, wherein the imaging data includes imaging type, protocol and/or technique information.

14. The method of claim 6, wherein the imaging data includes images whose acquisition technique was optimized for detecting a specific clinical condition.

15. The method of claim 6, wherein the knowledgebase includes a plurality of clinical conditions, and a plurality of associated algorithms and a plurality of algorithm parameters for the plurality of clinical conditions.

16. A system for enhancing visualization of clinical conditions comprising:
an input for receiving imaging data on a subject from an imaging modality;
a user interface for receiving user input on at least one suspected clinical condition of the subject undergoing imaging on an imaging modality; and
a processor coupled to the input and the user interface for processing the imaging data in association with a knowledgebase using an optimal image processing algorithm to enhance visualization of the at least one suspected clinical condition in at least one image.

17. The system of claim 16, further comprising a display coupled to the processor for displaying the enhanced visualization of the at least one suspected clinical condition in the at least one image.

18. The system of claim 16, further comprising a second input coupled to the processor for receiving clinical data on the subject.

19. The system of claim 18, wherein the processor includes at least one storage device for storing the clinical data, the imaging data and the knowledgebase.

20. The system of claim 16, wherein the processor is coupled to a network.

21. The system of claim 20, further comprising at least one picture archiving and communication system (PACS) workstation coupled to the network for reviewing the enhanced visualization of the at least one suspected clinical condition in at least one image.

22. The system of claim 16, wherein the input, the user interface and the processor comprise an acquisition workstation.

23. A system for enhancing visualization of clinical conditions comprising:
an acquisition workstation coupled to and receiving imaging data on a patient from an imaging modality, the acquisition workstation including a user interface for performing on-demand selection of at least one clinical condition to be enhanced in at least one image, and a computer coupled to the input and the user interface with at least one computer-usable medium having computer readable instructions stored thereon for execution by a processor, the computer performing a method comprising:
accessing clinical data on the patient undergoing imaging;
receiving imaging data from the imaging modality; and
processing the clinical data and the imaging data in association with a knowledgebase using an optimal image processing algorithm with optimal parameter settings to enhance visualization of a selected clinical condition in an image.

24. The system of claim 23, wherein the acquisition workstation is coupled to a network.

25. The system of claim 24, further comprising at least one picture archiving and communication system (PACS) workstation coupled to the network for reviewing the enhanced visualization of the selected clinical condition in the image.

26. The system of claim 23, wherein the acquisition workstation includes a display for reviewing the enhanced visualization of the selected clinical condition in the image.

27. The system of claim 26, wherein the display displays a list of clinical conditions to select from.

28. A computer program product for use with a computer, the computer program product comprising a computer-usable medium having computer readable instructions stored thereon for execution by a processor, the computer readable instructions comprising:
an accessing routine for accessing clinical data on a subject undergoing imaging on an imaging modality;
a receiving routing for receiving imaging data on the subject from the imaging modality; and
a processing routine for processing the clinical data and the imaging data in association with a knowledgebase using an optimal image processing algorithm with optimal parameter settings to enhance visualization of at least one clinical condition in at least one image.
